(19) United States
(12) Patent Application Publication
(10) Pub. No.: US 2026/0123921 A1
(43) Pub. Date: May 7, 2026

(54) MULTI-LUMEN SURGICAL TEXTILE

(71) Applicant: Arthrex, Inc., Naples, FL (US)

(72) Inventors: Matthew R. Herrington, Naples, FL (US); Nirav Patel, Naples, FL (US)

(73) Assignee: Arthrex, Inc., Naples, FL (US)

(21) Appl. No.: 19/372,975

(22) Filed: Oct. 29, 2025

Related U.S. Application Data
(60) Provisional application No. 63/714,984, filed on Nov. 1, 2024.

(51) Int. Cl. A61B 17/04

(52) U.S. Cl. CPC A61B 17/0401; A61B 2017/0404; A61B 2017/0406; A61B 2017/0409

(57) ABSTRACT

A surgical textile includes a plurality of continuous strands braided along a length. The continuous strands traverse a path along the length defining a plurality of tubular structures. The tubular structures form a plurality of lumens extending in parallel along the length.

### CROSS-REFERENCE TO RELATED APPLICATIONS

This application claims priority under 35 U.S.C. § 119(e) and the benefit of U.S. Provisional Application No. 63/714,984 entitled MULTI-LUMEN SURGICAL TEXTILE, filed on Nov. 1, 2024, by Herrington, et al., the entire disclosure of which is incorporated herein by reference.

### TECHNOLOGICAL FIELD

The disclosure generally relates to a textile construct for surgical applications.

### SUMMARY

The disclosure generally relates to a textile construct for surgical applications. In various implementations, the textile construct is provided in the form of a braided textile that may include one or more tubular structures. The tubular structures are formed from a plurality of continuous strands braided along a length. Along the length, the tubular structures may vary in cross section by size, diameter, thickness, or shape. Further, by implementing advanced braiding techniques, the tubular structures may transition along the length among flat sections, bifurcated sections, and merged sections, which may correspond to one common tubular section formed from two parallel tubular sections. As provided in the following disclosure, the various features of the braided textile may be implemented in combination with additional surgical implements to produce a variety of surgical constructs or assemblies to improve procedures.

### BRIEF DESCRIPTION OF THE DRAWINGS

FIG. 1 is a schematic diagram of a textile construct comprising a plurality of interlinked walls;

FIG. 2A is a cross-sectional view of a textile construct demonstrating a plurality of exemplary interconnected features;

FIG. 2B is a cross-sectional view of a textile construct demonstrating a plurality of exemplary interconnected features;

FIG. 2C is a cross-sectional view of a textile construct demonstrating a plurality of exemplary interconnected features;

FIG. 2D is a cross-sectional view of a textile construct demonstrating a plurality of exemplary interconnected features;

FIG. 2E is a cross-sectional view of a textile construct demonstrating a plurality of exemplary interconnected features;

FIG. 2F is a cross-sectional view of a textile construct demonstrating a plurality of exemplary interconnected features;

FIG. 3A is a perspective view demonstrating a textile construct comprising a plurality of centrally aligned interconnected features;

FIG. 3B is a perspective view of a textile construct demonstrating a plurality of interconnected features aligned along a common tangential plane;

FIG. 3C is a perspective view of a textile construct comprising a plurality of interlinked tubular segments arranged in a staggered or offset arrangement;

FIG. 4A is a perspective view of a textile construct demonstrating a plurality of tubular sections extending seamlessly to a common tapered section over a transition section;

FIG. 4B is a perspective view demonstrating a textile construct comprising a plurality of tubular structures transitioning into a common tubular structure;

FIG. 4C is a perspective view of a textile construct demonstrating a plurality of tubular structures transitioning into a flattened section and further transitioning into bifurcated, tapered sections;

FIG. 4D is a perspective view of a textile construct demonstrating an intermediate flat section interposed between a plurality of tubular sections;

FIG. 5 is a perspective view of a tensionable sheath comprising a textile construct;

FIG. 6 is a perspective view of a textile construct forming an anchor or button comprising a plurality of eyelets;

FIG. 7 is a perspective view of a textile construct comprising a plurality of tubular sections forming a sheath for a soft anchor apparatus;

FIG. 8 is a perspective view of a textile construct forming a compressible button or pledget; and

FIGS. 9A, 9B, and 9C are perspective views demonstrating a textile construct and exemplary process for forming an interconnected lumen from parallel tubular features in accordance with the disclosure.

### DETAILED DESCRIPTION

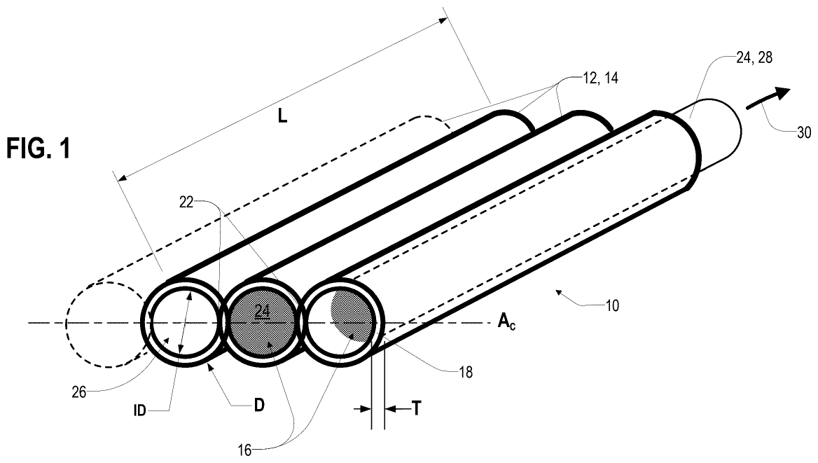

Referring generally to FIGS. 1-9, the disclosure may be implemented to include various textile constructs 10 that may provide for one or more interconnected features 12 for various surgical constructs or assemblies. As generally demonstrated in FIG. 1A, the textile construct 10 may comprise a plurality of tubular structures 14 that may form adjacent lumens 16 extending along a length L. In the example shown, three adjacent tubular structures 14 are interlinked, such that the neighboring lumens 16 are connected by and/or share adjacent walls 18. The tubular walls 18 may be formed by intertwined or braided fibers or strands to a thickness T formed between an outside diameter D and an inside diameter ID. The thickness T of the walls 18 and the corresponding diameters D, ID may vary based on the interlinked configuration or strand thickness of the material or layering of the strands forming the textile construct 10. For example, each of the tubular structures 14 may be formed by one or more layers of interlinked strands that may be manufactured through various techniques. For example, in a braided configuration, each of the tubular structures 14 may be formed by navigating the corresponding strands along complex, interlinked paths along which bobbin carriers may dispense each of the strands in coordination. In this way, the proportions, thickness, and/or materials associated with the tubular structures 14 and other interconnected features 12 may vary along the length L of the textile construct 10.

As demonstrated in FIG. 1, the strands forming the tubular structures 14 may travel along an interlinked path that may form an extended figure “8” or series of interlinked loops 22 that interconnect the tubular structures 14 along the tubular walls 18. In some implementations, the tubular structures 14 may include a core 24 or a hollow core 26 (e.g., a hollow interior or open lumen). In some implementations, the solid core 24 may be interlinked with the tubular wall 18. In other cases, the core 24 may correspond to an insert 28 that may be closely fit within the tubular wall 18 forming the tubular structure 14 or removable, depending on the application of the textile construct 10. An example of a removable insert 28 is illustrated by an arrow 30. As shown, the parallel, interconnected features 12 demonstrated in FIG. 1 include three adjacent tubular structures 14. However, it shall be understood that additional tubular structures 14 or other adjacent segments or features may be implemented as the interconnected features 12 as demonstrated in various examples discussed herein. Accordingly, the disclosure provides for the textile construct 10 in various configurations to suit a wide variety of applications.

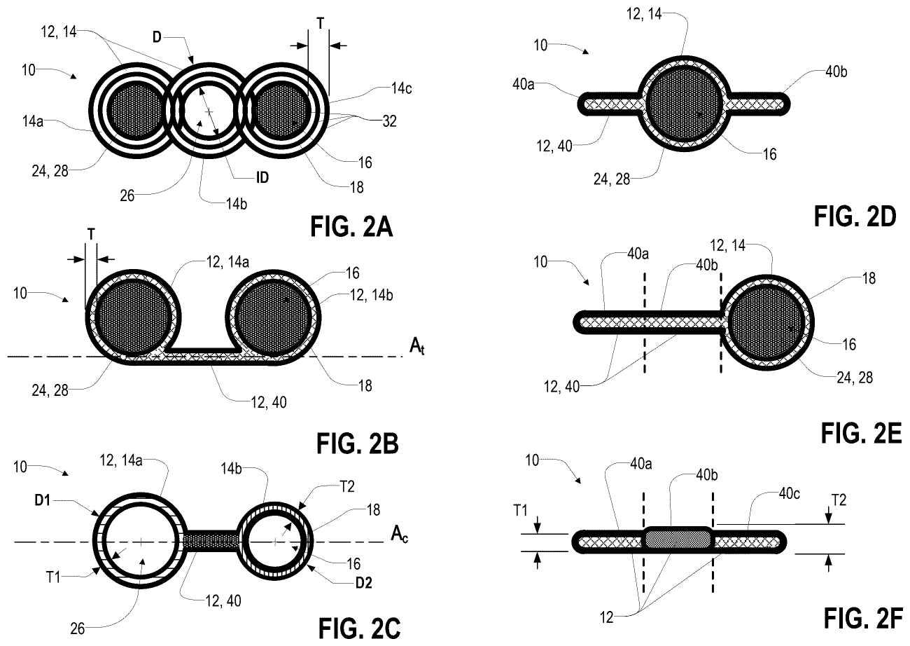

Referring now to FIGS. 2A-2F, various examples of the textile construct 10 are shown demonstrating the interconnected features 12 in a variety of configurations and combinations. Referring first to FIG. 2A, an example of the textile construct 10 is shown demonstrating the tubular walls 18 of the tubular structures 14 formed by multiple interlinked strand layers 32. As previously discussed, the thickness T of the tubular walls 18 may vary based on a thickness of the interlinked or braided strands and/or an additive thickness formed by a combination of strands forming the interlinked strand layers 32. As demonstrated from the cross-section representations, the outer diameter D of one or more of the tubular walls 18 may be attributed to a path of each of the strands forming the tubular walls 18. The inner diameter ID may similarly be designated based on the inner-most path of the strands forming each of the tubular structures 14. The thickness T formed between the outer diameter D and the inner diameter ID may be set based on the thickness of the strands or the number of interlinked strand layers 32 formed between an exterior surface 34 of the textile construct 10 and an interior surface 36 of each of the tubular walls 18 forming the lumens 16. Though demonstrated as concentric rings in the representative diagram in FIG. 2A, it shall be understood that the interlinked strand layers 32 may be braided or otherwise interconnected. Further, the tubular walls 18 may be formed with a consistent, strand spacing or variable spacing, depending on the application.

As further demonstrated in FIG. 2A, the plurality of tubular structures 14 comprises a first tubular section 14a, a second tubular section 14b, and a third tubular section 14c. The first and third tubular sections 14a, 14c include cores 24 (e.g., solid or semi-flexible), while the second tubular section 14b or intermediate section has a hollow core 26. The hollow core 26 of the intermediate, second tubular section 14b may provide for lateral flexibility, while the cores 24 or inserts 28 of the first and third tubular sections 14a, 14c may form stiffening shoulders on opposing sides of the hollow core 26. In general, the insert 28 forming the cores 24 may be formed from various materials including memory materials or materials that may be bent or formed along a longitudinal path to form one or more bends, angles, hooks, u-shapes, or other shapes along the length of the textile construct 10.

FIG. 2B demonstrates an implementation of the textile construct 10 incorporating one of the interconnected features 12 as a flat section 40. As described in various implementations, the flat section 40 may correspond to one or more strands that may otherwise be implemented in the tubular walls 18 that are braided and/or interconnected in a tape-like or ribbon-like construct. For example, the flattened section 40 may seamlessly extend from the interlinked paths forming the tubular structures 14 as previously discussed. Similar to other interconnected features 12 discussed herein, the flat section 40 may be interlinked with one or more of the tubular structures 14 via a transition section and/or one or more secondary manufacturing processes that may trim excess strands or join portions of the textile construct 10. In general, the various interconnected features 12 discussed herein may result primarily from automated manufacturing processes programed to define varying paths of the strands extending along the length L of the textile construct 10.

Still referring to FIG. 2B, the flat section 40 may extend along a plane P defined tangentially to the tubular walls 18 of the first tubular structure 14a and the third tubular structure 14c. As demonstrated in FIG. 2C, the flat section 40 may extend centrally through the lumens 16 of the tubular structures 14. For example, the lumens 16 defined by the tubular structures 14 may extend along a plane between central axes of two or more of the lumens 16. Additionally, the first and third tubular structures 14a, 14c shown in FIG. 2C may vary in diameter D1, D2 as well. As shown, the first diameter D1 is greater than the second diameter D2. Similarly, the thickness T of the tubular wall 18 forming the first tubular structure 14a is greater than the thickness T of the tubular wall 18 forming the second tubular structure 14b. As shown, the textile construct 10 in FIG. 2C includes three distinct interconnected features 12. A first interconnected feature may correspond to an enlarged tubular structure 14a having a first diameter D1. A second interconnected feature is represented by the second tubular structure 14b and having a smaller second diameter D2. Further, a third interconnected feature may correspond to the flat section 40 connecting the two tubular structures 14a, 14b.

In the examples shown, each of the interconnected features 12 may be formed from continuous, uniform strands extending along the length L. However, the interconnected features 12 may also be formed from various materials that may differ among the adjacent sections or along the length L. For example, one or more of the interconnected features 12 may comprise strands formed of different materials or one or more stiffening strands interlinked or braided within the textile construct 10. Accordingly, the disclosure may provide for the textile construct 10 to incorporate various interconnected features 12 that may be formed from a variety of materials, interlinked patterns, and thicknesses that may adjust the proportions, strength, and/or rigidity of the textile construct 10 to suit various applications.

Referring now to FIG. 2D, the textile construct 10 may include a plurality of flat sections 40. In the example shown, the textile construct 10 includes a first flat section 40a and a second flat section 40b positioned on opposing sides of a central tubular section 14. Each of FIGS. 2E and 2F demonstrate variations of the textile construct 10 including two flat sections 40a, 40b in FIG. 2E and three flat sections 40a, 40b, and 40c in FIG. 2F. As demonstrated in FIG. 2E, a tubular structure 14 may be formed on one side of the textile construct 10 with one or more flat sections 40a, 40b extending as an elongated flap. As shown in FIG. 2F, the plurality of flat sections 40a, 40b, 40c may vary in thickness T. For example, the first flat section 40a and the third flat section 40c may form a first thickness T1 and the intermediate, second flat section 40b may form a second thickness T2. The second thickness T2 may be greater than the first thickness T1. Additionally, though only represented as two thicknesses, each of the interconnected features 12 forming the textile construct 10 may differ in thickness and material, which may allow the textile construct 10 to provide for superior customization for a variety of applications. Similar to the thickness of the flat sections 40 demonstrated in FIG. 2F, the tubular sections 14a, 14b of FIG. 2C are also depicted having first and second thicknesses T1, T2 of the tubular walls 18.

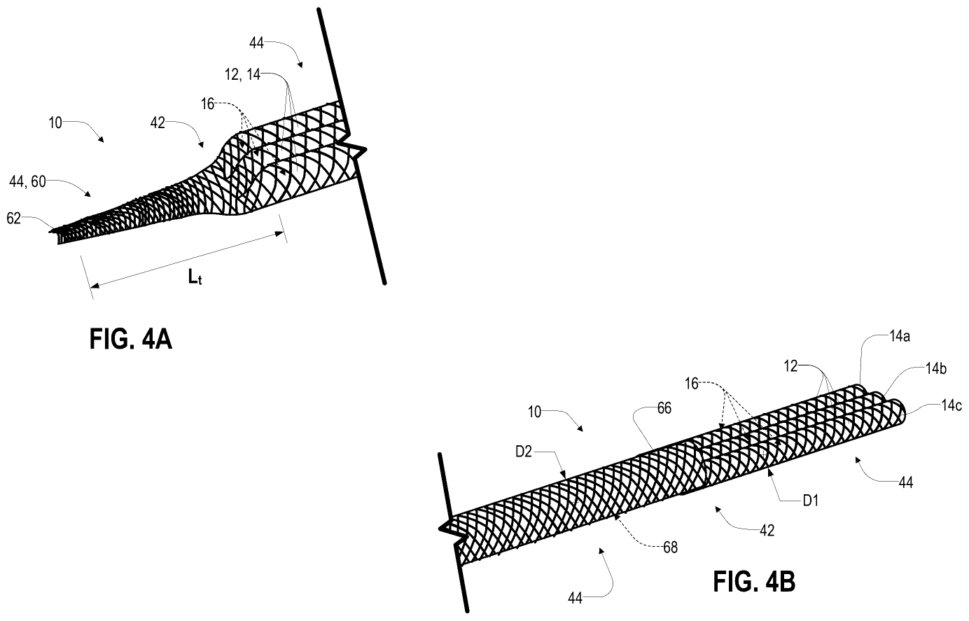

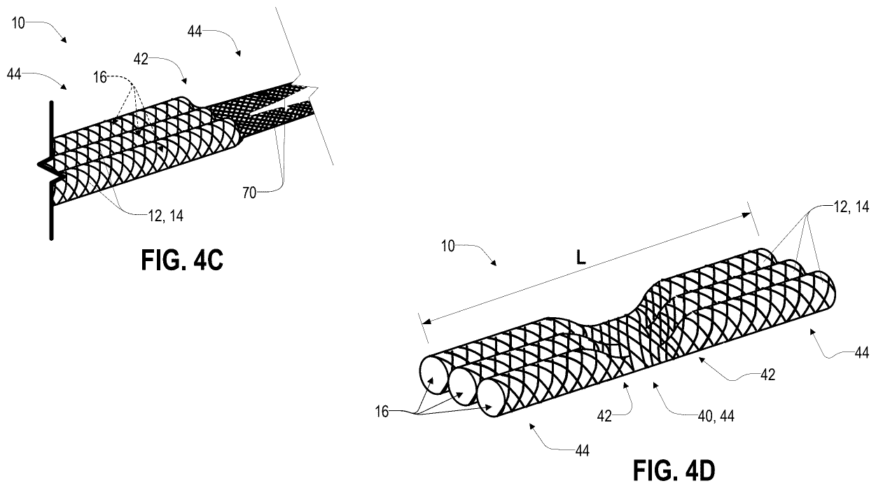

As later discussed in reference to FIGS. 4A-4D, the cross sections of each of the interconnected features 12 demonstrated in FIGS. 2A-2F may also vary along the length L of the textile construct 10 as longitudinal features 44. In some implementations, one or more automated textile manufacturing processes, for example, automatic braiding processes, may be programmed to direct the paths of the corresponding strands to vary along one or more transition sections 42 along the length L between the interconnected features 12 discussed herein. Accordingly, the disclosure may provide for the textile constructs 10 to provide for the various interconnected features 12 and longitudinal features 44, which may vary seamlessly along the length L of the textile constructs 10 to provide improved quality while offering the benefits provided by the various features 12 discussed herein.

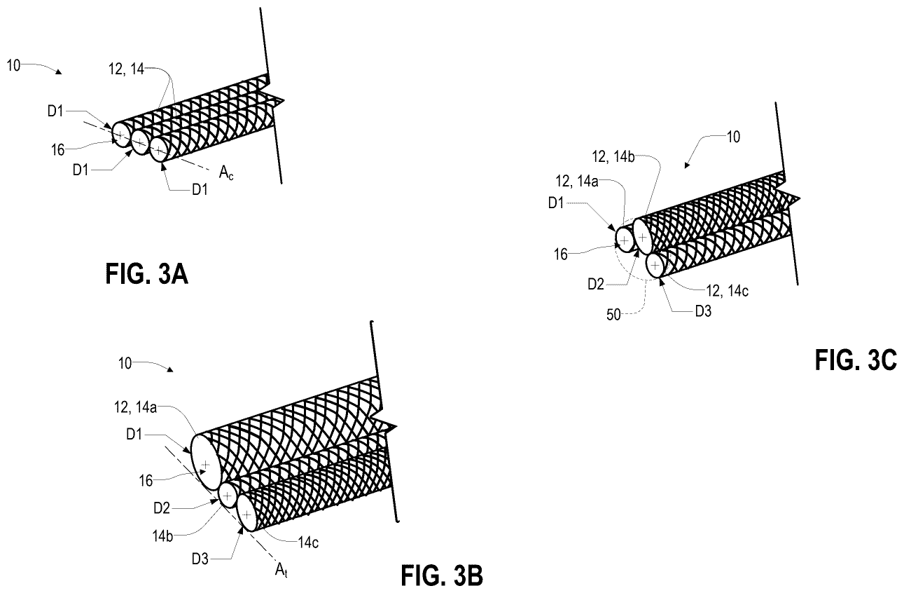

Referring now to FIGS. 3A-3C, additional examples of the interconnected features 12 forming the textile construct 10 are shown. In general, the examples of FIGS. 3A-3C demonstrate variations in the alignment and/or proportions of tubular structures 14 forming the interconnected features 12. As shown in FIG. 3A, the tubular structures 14 may have a common diameter D1. Additionally, a central axis A_{c} of the textile constructs 10 may extend through a cross-sectional center (e.g., round, elliptical, square, or rectangular) of each of the tubular structures 14. In such implementations, the lumens 16 formed by the tubular walls 18 may be commonly aligned along the transverse central axis A_{c}.

As shown in FIG. 3B, the center of the circular or elliptical cross sections forming the tubular structures 14 may also be aligned tangentially along the exterior surface 34 of the tubular walls 18. In this way, interconnected features 12 of differing proportions may be commonly aligned along a tangential axis Aₜ, such that the exterior surfaces 34 engage neighboring objects along a common planar surface. In the example shown in FIG. 3B, the first tubular structure 14a has a first diameter D1, the second tubular structure 14b has a second diameter D2, and the third tubular structure 14c has a third diameter D3. Each of the first, second, and third diameters D1, D2, D3 may vary in proportion, such that the corresponding tubular walls 18 and/or lumens 16 may be formed to provide the desirable features of longitudinal stiffness of the textile construct 10 and/or accommodating proportions within the passages forming the lumens 16. In the tangentially aligned configuration shown, the exterior surfaces 34 formed by the tubular walls 18 may be aligned regardless of variations in proportions among the diameters D1, D2, D3.

Referring now to FIG. 3C, the tubular structures 14 are demonstrated in a stacked or offset configuration. In contrast with the examples of FIGS. 3A and 3B, the stacked configuration of FIG. 3C may provide for a clearance profile 50 that may be better suited to pass through rounded or evenly proportioned openings through which the tape-like or ribbon-like proportions of alternative implementations of the textile construct 10 may pass less easily. Additionally, the positions and/or proportions of each of the interconnected features 12 may be radially positioned or staggered, such that the openings forming the lumens 16 may receive one or more objects (surgical constructs, tools, implements). The positions of the tubular structures may ensure that corresponding objects housed in the lumens 16 are positioned based on the relationship among the tubular structures 14. In this way, the relative positions of the tubular structures 14 may ensure that the objects are spaced in a controlled relationship that may correspond to their individual and/or mutual functions for a surgical procedure. For example, the spacing and/or positions, whether aligned along the central axis A_{c}, the tangential axis Aₜ or staggered, may ensure that the positions of the lumens 16 and the objects positioned therein are arranged in a known or intended relative position and spacing. Further, the relative positions of the lumens 16 may house the objects in a predetermined or functional relationship, which may provide for organization among the multiple objects in a surgical procedure. Accordingly, the alignment and proportions of the tubular structures 14 and various interconnected features 12 may be customized within the textile construct 10 to serve a variety of applications.

Referring now to FIGS. 4A-4D, variations of the textile constructs 10 are shown demonstrating various interconnected features 12, transition sections 42, and longitudinal features 44. As shown in FIG. 4A, the interconnected features 12 are tubular structures 14 including a tapered section 60 tapered over the transition section 42 to a narrow tail 62 or passing/threading section. In the example shown, the tubular walls 18 are first transitioned to the flat section 40, which is further tapered to the tail 62 along the tapered section 60 over a transition length Lₜ. The transition length Lₜ may be defined based on the interlinked path among the strands forming the textile construct 10. In some implementations, one or more strands of the textile construct 10 may be trimmed to reduce a bulk of the tail 62 to ensure the tail 62 easily passes through openings. In this configuration, the tail 62 may form a decreased cross section that may be better suited for passing through openings, tying, or connecting to various objects or constructs.

As shown in FIG. 4B, the tubular structures 14 may be interconnected with a common tubular structure 66. The common tubular structure 66 may correspond to common lumen 68 interconnected with each of the lumens 16 of the tubular structures 14. The transition section 42 between the common tubular structure 66 and the individual tubular structures 14 may be manufactured by adjusting the path of each of the interconnected or braided strands forming the tubular structures 14 to transition from the paths forming the tubular structures 14 to a common perimeter path forming the common tubular structure 66. The common tubular structure 66 may similarly house one or more objects, tools, accessories, etc. associated with a surgical procedure within the common lumen 68 and may provide for the organization of such objects via each of the lumens 16. Additionally, the common tubular structure 66 may correspond to a simplified construction of the strands that may be more readily adjusted over the transition length L_{T} to other arrangements of the interconnected features 12 or the longitudinal features 44. Accordingly, the common tubular structure may serve an intermediate, longitudinal feature 44 to transition to different features along the length of the textile construct 10.

Referring now to FIG. 4C, a textile construct 10 is shown demonstrating the tubular structures 14 comprising a transition section 42 to the flat section 40. In the example shown, the flat section 40 may be divided, forming bifurcated sections 70 of the textile construct 10 formed from the strands of the interconnected features 12. The bifurcated section 70 may be beneficial for interconnecting the textile construct 10 with one or more structures either in manufacturing a surgical construct or in practical applications for surgical procedures. For example, the bifurcated sections 70 may be readily tied or attached to one or more structures and may be more readily passed through openings to provide threading sections 62 similar to the tail 60.

As demonstrated in FIG. 4D, the longitudinal features 44 may be seamlessly integrated along the length L of the textile construct 10 in various sequences. For example, the textile construct 10 of FIG. 4D demonstrates the flat section 40 seamlessly formed along the length L of the textile construct 10 between two sections forming the tubular structures 14. Similarly, the cross-sectional diameter D of the tubular structures 14 may also vary seamlessly along the length L. Though demonstrated with each of the three tubular structures 14 forming the flat section 40, one or more of the tubular structures 14 may vary in cross section to form the interconnected features 12 previously discussed in reference to FIGS. 2A-2F or similar structures without departing from the spirit of the disclosure. As previously discussed, the cross sections of the longitudinal features 44 may be varied along the length L by adjusting the paths of the strands through one or more automated manufacturing processes. Additionally, manual processes may be implemented to trim, interconnect, or otherwise form the transition sections 42 to form the textile construct 10.

As previously described, each of the surgical constructs 80 may comprise one or more of the parallel interconnected features 12 and the longitudinal features 44 previously discussed in reference to FIGS. 2-4. In addition to the visually discernable characteristics previously discussed in reference to these figures, it shall also be noted that the constituent strands forming the textile constructs 10 may vary among each of the interconnected features 12, transition sections 42, and/or the longitudinal features 44. For example, in some implementations, the material type, flexibility, gage, and/or structure of each of the strands forming the textile constructs 10 may be selected to provide the corresponding flexibility, rigidity, wall thickness T, and/or other features necessary to implement the various constructs discussed herein. The materials forming the textile construct may correspond to polyester, silk, nylon, aramid, a long chain synthetic polymer, a bioabsorbable fiber, or various polymeric, metallic, or organic materials. Accordingly, the textile construct 10 may be implemented in a variety of ways to provide improved and customizable structures to suit the variety of procedures.

The cores 24 or inserts 28 may be formed from various materials and may serve to adjust the operational flexibility of one or more portions of the textile construct 10 relative to each other. For example, the cores 24 or inserts may adjust the longitudinal flexibility of the textile construct 10 along the length L and/or the transverse flexibility among the parallel interconnected features 12. For example, the inclusion of the core 24 or insert 28 in one of the lumens 16 may reduce the longitudinal flexibility of the corresponding tubular structure 14 while the remaining tubular structures 14 may flex or roll transverse to the tubular structure 14 comprising the core 24. Similarly, the incorporation of the cores 24 or inserts 28 in two lumens 16 may increase the longitudinal rigidity of two of the tubular structures 14 with a third tubular structure (e.g., 14b) bending more freely transverse to the first and/or second tubular structures (e.g., 14a, 14c). In cases where each of the lumens 16 include the cores 24 or inserts 28, the resulting textile construct 10 may bend and flex as a significantly unitary body with a common longitudinal flexibility along the length L. Finally, the cores 24 or inserts 28 may be included in the lumens 16 along the entire length L or a portion of the length L. The portion of the length including the core 24 or insert 28 may correspond to a length including one or the longitudinal features 44 as discussed in reference to FIGS. 4A-4D.

The material forming the cores 24 or inserts 28 may include various materials. For example, one or more strands of wire, thread, and/or fabric may be included in one or more of the lumens 16. In some cases, a solid or semi-rigid core may be enclosed within one or more tubular structures 14, which may correspond to polymeric or metallic inserts. In some implementations, a padding or stuffing may be enclosed within one or more tubular structures 14, which may limit or reduce the longitudinal flexibility and may also serve as a cushion or fill that may increase or maintain volumetric proportions of the corresponding tubular structure 16 when deployed. The cores 24 or inserts 28 may be incorporated as one or more preformed shapes or bendable materials that may maintain a desired shape (e.g., malleable steel, memory alloys, etc.). The cores 24 or inserts 28 may also include a combination of multiple materials (e.g., a padding layer, a stiffening layer, stiffening strands within a malleable core, etc.). Accordingly, the textile construct 10 may be constructed to provide a variety of functional characteristics to suit a wide range of applications.

Though demonstrated and described in FIG. 4C as first transitioning to a flat section, in some implementations, one or more of the plurality of tubular structures 14 may transition directly to the bifurcated section 70 from the tubular section. For example, the continuous strands of the tubular wall forming one or more of the tubular structures 14 may divide into neighboring parallel braids forming the bifurcated section 70. In some implementations, the neighboring parallel braids may correspond to flat braids, round braids, or various cross-sectional shapes. In various cases, the bifurcated sections 70 formed from the tubular walls may form an eyelet defining an opening into the at least one lumen formed by the corresponding to the tubular structure 14 from which the bifurcated section 70 is formed. In this way, the bifurcated section 70 may provide access into the lumen for one more sutures or fibers of a surgical construct.

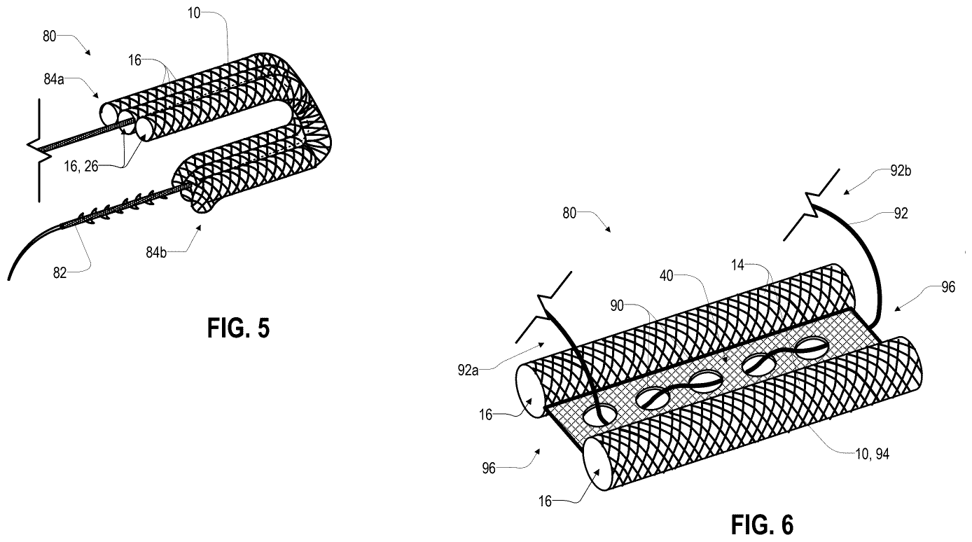

Referring generally to FIGS. 5-9, examples of textile construct 10 are shown implemented forming the various of surgical constructs 80. Referring to FIG. 5, a textile construct 10 is shown having a barbed suture 82 extending through one of the lumens 16 from a proximal end portion 84a to a distal end portion 84b. In the example shown, the barbed suture 82 may extend through a central lumen 16 of the tubular structure 14 from the proximal end portion 84a and pass through the corresponding tubular wall 18 at the distal end portion 84b. In this configuration, the barbed suture 82 may pass directionally through the interlinked strands forming the textile construct 10 from the proximal end portion 84a to the distal end portion 84b. Further, in response to the application of tension to the barbed suture 82 at the proximal end portion 84a, the barbed suture 82 may bind on the strands forming the textile construct 10, such that the textile construct 10 collapses or folds. The collapsing or folding of the textile construct 10 may bind or hold textile construct 10 against a surface or bunched within an opening, thereby securing the distal end portion 84b of the surgical construct 80. In this way, the textile construct 10 may better secure the surgical construct 80 for use in a surgical procedure.

Referring now to FIG. 6, the interconnected features 12 forming the textile construct 10 may include a flat section 40 formed between two of the tubular structures 14. Additionally, a connection interface comprising one or more eyelets 90 or openings may be formed through the flat section 40 as longitudinal features 44 along the length L of the textile construct 10. In the example shown, a suture 92 extends through the eyelets 90 between a proximal end portion 92a and a distal end portion 92b. In this configuration, the textile construct 10 may form an anchor or button 94 against which tension may be applied to the proximal and distal ends 92a, 92b of the suture 92 to support the surgical construct 80. As a result of the tension applied to the suture 92, opposing end portions 96 of the textile construct 10 may collapse or fold, bunching the portions of the flat section 40 between the eyelets 90. The bunching of the flat section 40, which may serve to retain or bind the textile construct 10 against a tissue or a structure associated with a surgical procedure.

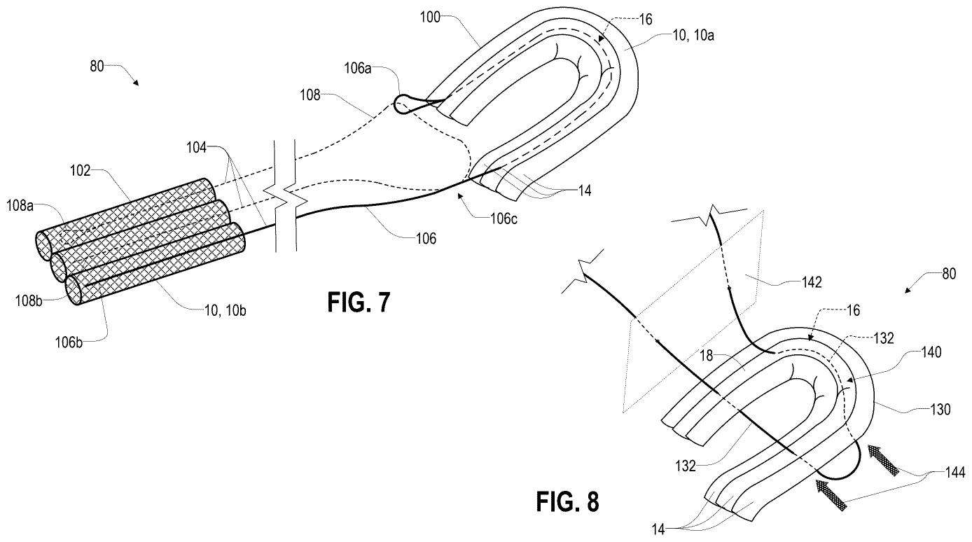

As demonstrated in FIG. 7, a plurality of the textile constructs 10 may be implemented in a common surgical construct 80. As shown, a first textile construct 10a may be implemented as a soft anchor 100 and a second textile construct 10b may form a sheath 102 or suture sock within which the end portions of multiple loose ends 104 or loose portions of the surgical construct 80 may be arranged and housed. In the example shown, the loose ends 104 corresponding to ends of a connecting suture 106 and a passing suture 108. In operation, the sheath 102 may retain the loose ends in a known or planned arrangement making them accessible and organized for access during a surgical procedure. In various implementations, the sheath 102 may serve as packaging to prevent tangling among the loose ends 104 or portions allowing the surgical construct 80 to be deployed readily.

The surgical construct 80 shown in FIG. 7 may correspond to a knotless suture assembly. The connecting suture 106 may extend through one of the lumens 16 of the soft anchor 100. Though demonstrated as passing through a central one of the lumens 16, the connecting suture 106 may extend through one or more of the lumens 16 formed by the tubular structures 14. On opposing ends, the connecting suture 106 forms a connecting eyelet 106a and a connecting strand 106b. Each of the opposing ends of the connecting suture 106 may pass through the tubular wall 18 of the soft anchor 100 near ends separated by a portion of the length L. A passing suture 108 of the surgical construct 80 may extend from a passing eyelet 108a to a passing strand 108b on opposing ends. Along its length, the passing suture 104b may extend through the connecting eyelet 106a and through a splice region 106c of the connecting suture 106, thereby forming the knotless suture construct with the soft anchor 100.

Still referring to FIG. 7, the loose ends 104 of the sutures 106, 108 may include the connecting strand 106b, the passing eyelet 108a, and the passing strand 108b. Each of the loose ends 104 of the construct 80 may be enclosed or housed within the tubular structures 14 forming the lumens 16 of the sheath 102. In operation, the sheath 102 may slidably retain the loose ends 104 of the sutures 104a, 104b under light compression within the lumens 16 until the sutures 106, 108 are deployed in a surgical procedure. In this way, the sheath 102 may maintain the organization and arrangement among the loose ends 104 of the sutures 106, 108 until they are deployed in a surgical procedure. Accordingly, the textile construct may be implemented to limit preparation time and ensure that portions of the surgical construct 80 are packaged and presented to surgeons in an organized and consistent arrangement.

Referring now to FIG. 8, the textile construct 10 is demonstrated as a compressible pledget 130 forming the surgical construct 80 in combination with at least one suture 132. In the example shown, the suture 132 extends through opposing tubular walls 18 of one of the tubular structure 14. As shown, the textile construct 10 may be folded, such that suture 132 extends to opposing sides 134 of the textile constructs 10 in a folded configuration 136. In this configuration, the suture 132 may extend through a common exterior surface 34 of one or more of the tubular walls 18 on the opposing side 134 of the textile construct 10 in the folded configuration 136. As previously discussed, the textile construct 10 may comprise one or more cores 24 or inserts 28, which may provide for stiffening or retention of textile construct 10 in a pre-bent or preformed shape, (e.g., folded approximately in half, bent) to assist in aligning the compressible pledget 130 for deployment.

The construct 80 of FIG. 8 may include one or more sections of the suture 132 extending through one of the lumens 16 to form a sheathed section 140 of the suture 132. The sheathed section 140 may extend to the tubular wall 18 on opposing sides, wherein the suture 132 may extend through the textile construct 10. In this configuration, the textile construct 10 may collapse and bunch about the sheathed section 140 as a result of tension applied to the suture 132. As shown, the tension of the suture 132 may be drawn against a structure 142 (e.g., a tissue surface, implant surface, etc.), such that the portions of the suture 132 passing through the tubular walls 18 and/or lumens 16 of the textile construct 10 may compress as demonstrated by the arrows 144. As a result, the overlapping exterior surfaces 34 formed by the folded configuration 136 may collapse and compress against the structure 142, thereby providing a strong anchor for the suture 132.

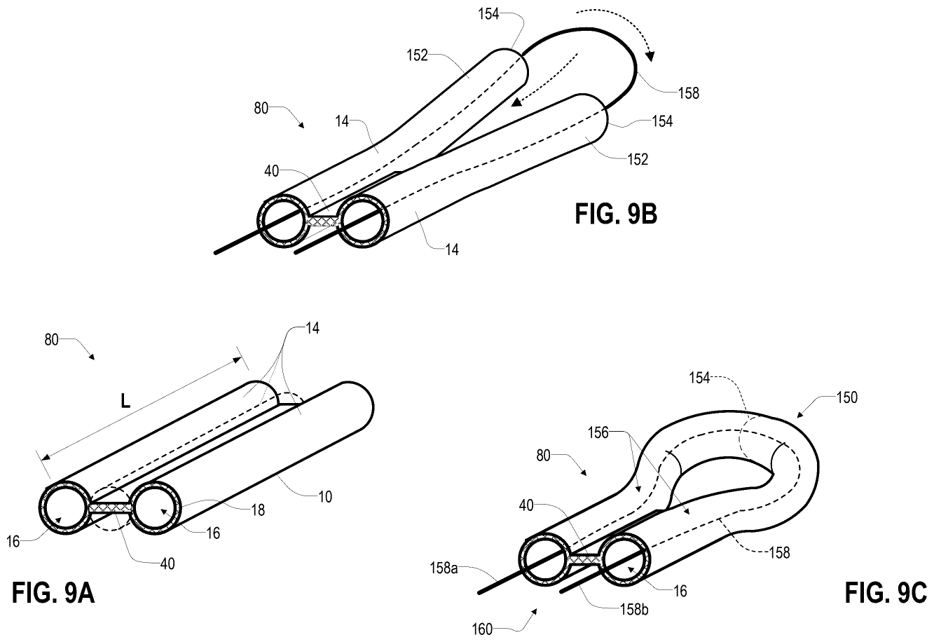

Referring now to FIGS. 9A-9C, yet another implementation of a surgical construct 80 comprising the textile constructs 10 is shown. In general, FIGS. 9A-9C demonstrate a process for providing a joined loop 150 with two of the tubular structures 14 of the textile construct 10. As demonstrated in FIG. 9A, the interconnected features 12 of the textile construct 10 may comprise two, three, or more of the tubular structures 14 extending along the length L. In the example shown, two of the tubular structures 14 extend with a flat section 40 therebetween. As demonstrated in FIG. 9B, a portion of the flat section 40 may be trimmed or cut back, leaving the tubular structures 14 as free ends 152 on one side of the textile construct 10. As a result, the free ends 152 may be separated and moved relative to each other at the free end. As shown in FIG. 9C, the free ends 152 of the tubular structures 14 may be joined or fused along a splice section 154. In this configuration, the free ends 152 of the tubular structures 14 may form a joined loop 150. The joined 150 loop may provide an overlapping interior passage 156 through which one or more objects, in this case a suture 158, may extend. The passage of the suture 158 through the joined loop 150 may ensure that the opposing ends of the suture 158a, 158b are accessible at a common end 160 of the textile construct 10.

The materials forming the textile construct may correspond to continuous or semi-continuous strands or filaments. In general, the strands or filaments may be formed from natural or synthetic materials depending on the application. Examples of natural materials include catgut, silk, and cotton. Synthetic materials include nylon, polypropylene, polybutester, and stainless steel. Further, some materials associated with the strands or filaments of the textiles or structures disclosed include absorbable materials and non-absorbable materials. Examples of absorbable materials include PDS (polydioxanone), Vicryl (glycolide and lactide), polyglactin 910, polyglycolic acid, poliglecaprone, polyglycolide-trimethylene carbonate, polyglycone 6211, and glycomer 631 are examples of absorbable sutures. Non-absorbabe materials include silk, polybutester, braided polyester, nylon, polypropylene, polyhexafuropropylene, goretex, and stainless steel. In some implementations, one or more of the strands or filaments may be formed from a shape memory materials (SMMs) are materials that can return to their original shape after being deformed. Shape memory materials may include shape memory alloys or polymers including nitinol, Austenite, copper-aluminum-nickel, copper-zinc-aluminum, and iron-manganese-silico, polytetrafluoroethylene (PFTE), polylactide (PLA), and ethylene-vinyl acetate (EVA). Accordingly, the constructs discussed may be manufactured from a variety of materials.

In some implementations, the materials associated with the strands or the filaments may differ between the neighboring or interconnected features 12 forming the textile construct 10. For example, as previously discussed, the interlinked paths formed by the strands may be dispensed by bobbin carriers. In some implementations, the materials forming the strands or filaments wound on one or more of the bobbins and dispensed from the carriers may differ such that all of a portion of the strands or filaments forming the interconnected features 12 may differ along all or a portion of the length L of the textile construct 10. For example, all or a number and corresponding ratio of the strands or fibers forming the interconnected features 12 may vary in material type, thickness, density, etc. In this way, the proportions, thickness, and/or materials associated with the tubular structures 14 and other interconnected features 12 may cause the resulting textile construct 10 to vary longitudinal stiffness or flexibility along the length or lateral stiffness and flexibility along a width or height. Accordingly, the disclosure may provide for the textile construct 10 to incorporate various interconnected features 12 that may be formed from a variety of materials, interlinked patterns, and thicknesses that may adjust the proportions, strength, and/or rigidity of the textile construct 10 to suit various applications.

According to some aspects of the disclosure, a surgical textile includes a plurality of continuous strands braided along a length. The continuous strands traverse a path along the length defining a plurality of tubular structures forming a plurality of lumens and extending in parallel along the length.

According to various aspects, the disclosure may implement one or more of the following features or configurations in various combinations:
the continuous strands are braided in an extended FIG. 8 or interlinked loops;
a first tubular structure of the plurality of tubular structures comprises a core extending through the corresponding lumen;
the core is formed of at least one of a polymeric or metallic material;
the core is formed of a textile structure or stuffing fiber;
the textile structure comprises a braided structure;
the continuous strands forming the tubular structure are braided around the core;
the core comprises a shape memory material;
the core is a deformable material that retains a contour shape of the surgical textile construct along the length;
the plurality of tubular structures form adjacent walls formed by an intersection of the continuous strands interlinked between neighboring tubular structures;
the tubular structures braided from the continuous strands forming adjacent structures that vary in cross section by size, diameter, thickness, or shape;
at least one of the continuous strands of at least one of the plurality of tubular structures extends through a transition section along the length, wherein the transition section comprises a braided transition of the continuous strands from the at least one tubular structure to a flat section, a bifurcated section, or a merged section;
the flat section is formed from the continuous strands by interlinking a plurality of tubular wall strands into a flat-braided section;
the bifurcated section is formed from the continuous strands dividing a plurality of tubular wall strands into a plurality of neighboring parallel braids;
the neighboring parallel braids comprise flat braids or round braids;
the neighboring parallel braids of the bifurcated section form an eyelet defining an opening into the at least one lumen corresponding to the bifurcated section of the tubular structure;
the opening provides access to the lumen for one more sutures or fibers of a surgical construct; and/or
the merged section comprises a merged tubular structure or a merged braided structure formed from the continuous strands from two or more of the plurality of tubular structures.

According to another aspect of the disclosure, a method for forming a surgical textile includes braiding a plurality of continuous strands along a length by traversing a path along the length defining at a plurality of tubular structures. The tubular structures form a plurality of lumens and extend in parallel along the length.

According to various aspects, the disclosure may implement one or more of the following features or configurations in various combinations:
the path traversed by the continuous strands comprises intersects between neighboring sections of the tubular structures forming adjacent walls;
braiding the continuous strands of at least one of the plurality of tubular structures through a transition section along the length;
the transition section comprises a braided transition of the continuous strands from the at least one tubular structure to a flat section, a bifurcated section, or a merged section;
the flat section is formed from the continuous strands by interlinking a plurality of tubular wall strands into a flat-braided section;
the bifurcated section is formed from the continuous strands dividing a plurality of tubular wall strands into a plurality of neighboring parallel braids;
the neighboring parallel braids comprise flat braids or round braids;
forming an eyelet through defining an opening though at least one tubular wall of the tubular structure; and/or
the eyelet is formed by neighboring parallel braids of a bifurcated section from separated sides of the tubular structure.

According to yet another aspect of the disclosure, a surgical construct includes a braided textile comprising a plurality of continuous strands braided along a length. The continuous strands traverse a path along the length defining a plurality of tubular structures forming a plurality of lumens and extending in parallel along the length and a suture passing through a portion of the braided textile.

According to various aspects, the disclosure may implement one or more of the following features or configurations in various combinations:
the plurality of tubular structures include a first tubular structure and a second tubular structure interconnected by a flat section;
the flat section forms a plurality of eyelets or grommets from the continuous strands;
the suture extends through the eyelets;
the braided textile forms a pledget or anchor against which tension is applied to the suture, compressing at least one of tubular structures;
the plurality of tubular structures form sheaths configured to retain end portions of the suture; and/or
sheaths divide and separate the portions of the suture and one or more connected loops or passing needles.

As used herein, words of approximation such as, without limitation, “approximately,” “substantially,” or “about” refer to a condition that when so modified is understood to not necessarily be absolute or perfect but would be considered close enough to those of ordinary skill in the art to warrant designating the condition as being present. Further, the lack of such modifying terms does not otherwise require strict interpretation of the corresponding value or property. Instead, the extent to which the associated interpretation varies will depend on how great a change can be instituted and still have one of ordinary skill in the art recognize the modified feature as having the required characteristics or capabilities of the unmodified feature. Such determinations may vary considerably depending on the technological field based on the corresponding equivalency associated with the described operation or property. In general, but subject to the preceding discussion, a numerical value herein that is modified by a word of approximation such as “approximately” may vary from the stated value by ±0.5%, ±1%, ±2%, ±3%, ±4%, ±5%, ±10%, ±12%, or ±15%.

Any element in a claim that does not explicitly state “means” for performing a specified function or “step” for performing a specified function, should not be interpreted as a “means” or “step” clause as specified in 35 U.S.C. § 112.

It will be understood that any described processes or steps within described processes may be combined with other disclosed processes or steps to form structures within the scope of the present device. The exemplary structures and processes disclosed herein are for illustrative purposes and are not to be construed as limiting.

1. A surgical textile comprising:
a plurality of continuous strands braided along a length, wherein the continuous strands traverse a path along the length defining a plurality of tubular structures forming a plurality of lumens and extending in parallel along the length.

2. The surgical textile according to claim 1, wherein the continuous strands are braided in interlinked loops.

3. The surgical textile according to claim 1, wherein a first tubular structure of the plurality of tubular structures comprises a core extending through the corresponding lumen.

4. The surgical textile according to claim 1, wherein the core is formed of at least one of a polymeric or metallic material.

5. The surgical textile according to claim 1, wherein the plurality of tubular structures form adjacent walls formed by an intersection of the continuous strands interlinked between neighboring tubular structures.

6. The surgical textile according to claim 1, wherein at least one of the continuous strands of at least one of the plurality of tubular structures extends through a transition section along the length, wherein the transition section comprises a braided transition of the continuous strands from the at least one tubular structure to a flat section, a bifurcated section, or a merged section.

7. The surgical textile according to claim 6, wherein the bifurcated section is formed from the continuous strands dividing a plurality of tubular wall strands into a plurality of neighboring parallel braids.

8. The surgical textile according to claim 7, wherein the neighboring parallel braids comprise flat braids or round braids.

9. The surgical textile according to claim 8, wherein the neighboring parallel braids of the bifurcated section form an eyelet defining an opening into the at least one lumen corresponding to the bifurcated section of the tubular structure.

10. The surgical textile according to claim 9, wherein the opening provides access to the lumen for one more sutures or fibers of a surgical construct.

11. The surgical textile according to claim 6, wherein the merged section comprises a merged tubular structure or a merged braided structure formed from the continuous strands from two or more of the plurality of tubular structures.

12. A method for forming a surgical textile comprising:
braiding a plurality of continuous strands along a length by traversing a path along the length defining at a plurality of tubular structures, wherein the tubular structures form a plurality of lumens and extend in parallel along the length, wherein the path traversed by the continuous strands comprises intersects between neighboring sections of the tubular structures forming adjacent walls.

13. The method according to claim 12, further comprising:
braiding the continuous strands of at least one of the plurality of tubular structures through a transition section along the length.

14. The method according to claim 13, wherein the transition section comprises a braided transition of the continuous strands from the at least one tubular structure to a flat section, a bifurcated section, or a merged section.

15. The method according to claim 14, wherein the flat section is formed from the continuous strands by interlinking a plurality of tubular wall strands into a flat-braided section.

16. The method according to claim 14, wherein the bifurcated section is formed from the continuous strands dividing a plurality of tubular wall strands into a plurality of neighboring parallel braids.

17. The method according to claim 16, wherein the neighboring parallel braids comprise flat braids or round braids.

18. The method according to claim 12, further comprising:
forming an eyelet defining an opening through at least one tubular wall of the tubular structure, wherein the eyelet is formed by neighboring parallel braids of a bifurcated section from separated sides of the tubular structure.

19. A surgical construct comprising:
a braided textile comprising a plurality of continuous strands braided along a length, wherein the continuous strands traverse a path along the length defining a plurality of tubular structures forming a plurality of lumens and extending in parallel along the length; and
a suture passing through a portion of the braided textile.

20. The surgical construct according to claim 19, wherein the plurality of tubular structures comprise a first tubular structure and a second tubular structure interconnected by a flat section, wherein the flat section forms a plurality of eyelets or grommets from the continuous strands.
